(19) United States
(12) Patent Application Publication
(10) Pub. No.: US 2023/0228771 A1
(43) Pub. Date: Jul. 20, 2023

(54) DETERMINATION OF DISEASE-SPECIFIC PROTEIN AGGREGATES IN STOOL SAMPLES

(71) Applicant: FORSCHUNGSZENTRUM JUELICH GMBH, Juelich (DE)

(72) Inventors: Gueltekin TAMGUENEY, Duesseldorf (DE); Dieter WILLBOLD, Juelich (DE); Marlene PILS, Duesseldorf (DE); Oliver BANNACH, Duesseldorf (DE)

(21) Appl. No.: 17/998,691

(22) PCT Filed: May 25, 2021
(86) PCT No.: PCT/EP2021/063833

§ 371 Date: Nov. 14, 2022

(30) Foreign Application Priority Data
May 28, 2020 (DE) 10 2020 114 278.1

(51) Int. Cl. G01N 33/68; G01N 33/543; G01N 33/533; G01N 33/50

(52) U.S. Cl. CPC G01N 33/6896; G01N 33/543; G01N 33/533; G01N 33/5008; G01N 2800/2821

(57) ABSTRACT

The invention relates to methods for selective quantification of A-beta or alpha-synuclein aggregates comprising the immobiliZation of anti-A-beta or alpha-synuclein antibodies on a substrate, application of the stool sample to be tested to the substrate, addition of probes labelled for detection which by specific binding to A-beta or alpha-synuclein aggregates mark these and detection of the marked aggregates.

The invention relates to methods for the determination of disease-specific protein aggregates in stool samples, in particular based on the selective quantification of e.g. alpha-synuclein or amyloid-beta (A-beta) aggregates, comprising the immobilization of alpha-synuclein or A-beta capture molecules on a substrate, applying the sample to be tested to the substrate, adding probes labelled for detection which by specific binding to alpha-synuclein or A-beta aggregates mark these, and detecting the marked aggregates.

Characteristic for a plurality of diseases of the central nervous system is the occurrence of misfolded, aggregated proteins in the course of the disease. As examples Parkinson's disease and Alzheimer's disease are mentioned here. Parkinson's disease (PD, Latin=Morbus Parkinson) is characterized by a pathological accumulation of aggregated alpha-synuclein, whereas Alzheimer's disease (AD, Alzheimer's dementia, Latin=Morbus Alzheimer) is characterized by the pathological accumulation of aggregated A-beta and aggregated tau.

Neurodegenerative diseases such as AD and PD, for example, belong to a heterogeneous group of clinical conditions whose common criterion in many cases (but not exclusively) is aggregation and deposition of a respective specific protein in the ordered conformation of beta-sheet structure. AD and PD are an increasing problem in today's society, as more and more people are affected by it due to increased life expectancy, and the disease thus has an impact on social security systems and their financial viability.

Pathological aggregates, such as oligomers or fibrils, of endogenous proteins occur in many neurodegenerative diseases. In AD, for example, A-beta peptide deposits are found in the brain, and in PD, alpha-synuclein deposits. However, the A-beta-peptide deposits (or peptide fibrils) are merely the final stage of a process that begins with the cleavage of monomeric A-beta-peptides from APP (amyloid precursor protein), subsequently forms neurotoxic A-beta-peptide oligomers and finally or alternatively ends with A-beta-peptide fibrils. The main pathological feature of AD is the formation of senile or amyloid plaques consisting of the A-beta peptide and neurofibrillary deposits of the tau protein. The precursor protein of the A-beta peptide, APP, is localised in the cell wall of neurons. Through proteolytic degradation and subsequent modification, it gives rise to A-beta fragments of different lengths and types, such as A-beta 1-40 (A-beta 40), A-beta 1-42 (A-beta 42) or pGluA-beta 3-42. Such monomeric A-beta peptides are formed throughout the entire life, also in a healthy organism.

According to the amyloid cascade hypothesis from the 1990s, the A-beta deposits in the form of plaques are the triggers of the disease symptoms. In recent years, however, different studies indicate that especially the small, freely diffusing A-beta oligomers have the greatest toxicity and are responsible for the development and progression of AD. Thus, aggregates of A-beta peptide are directly linked to AD pathogenesis.

Neurodegenerative diseases are usually diagnosed by neurological examination. To support the diagnosis, imaging techniques such as positron emission tomography (PET) in AD or DaTscan in PD can be used in a few neurodegenerative diseases. Usually, laboratory diagnostic procedures such as ELISA detect disease-related concentration changes of one or more substances from body fluids such as CSF, blood, plasma, serum, saliva or urine. For the diagnosis of Alzheimer's disease, the ratio of A-beta 42 to A-beta 40 or the amount of phosphorylated tau can thus also be determined from cerebrospinal fluid samples using ELISA. However, an absolutely reliable diagnosis of a neurodegenerative disease is often only possible post-mortem through the histopathological detection of disease-specific protein aggregates in the central nervous system, for which tissue sections of the brain are required.

The neurological diagnosis of neurodegenerative diseases can often be incorrect in the early stages of these diseases or not recognise them at all because the symptoms of the disease are only mild at early stages of the disease. Imaging techniques such as PET or DaTscan are not universally accessible and are expensive, and only a few specific neurodegenerative diseases such as AD or PD can be diagnosed with imaging techniques. Most importantly, the collection of CSF and blood samples is invasive and the former is usually very painful for patients.

For example, the amounts of various substances in patients' blood or CSF have been studied and their usefulness as biomarkers analysed. One of these substances is the A-beta peptide. The most reliable method seemed to be the determination of the A-beta peptide content in the cerebrospinal fluid of patients over a period of 6-24 months. However, this requires several samples of cerebrospinal fluid over a long period of time.

Despite these different approaches, no reliable biomarker has yet been established. Another complicating factor is that only a few detection systems are available for the specific quantification of A-beta aggregates in distinction to A-beta monomers and/or the total A-beta content. ELISAs, in which the A-beta oligomers are detected by antibodies, are being used as one possible detection system so far. The antibodies used in these either only recognise very specific types of A-beta oligomers or non-specifically other oligomers that do not consist of A-beta peptides but of completely different proteins, which has a disadvantageous effect on the evaluation.

Sandwich ELISA measurements serve as a further detection method. Here, A-beta-specific antibodies are used to immobilise the A-beta oligomers. The same antibodies are subsequently used for detection. Monomers do not lead to any signal according to this method, because the antibody binding site is already occupied by the capture molecules. Specific signals are thus only generated by dimers or larger oligomers. In the evaluation, however, such a method only allows the quantification of the sum of all aggregates present in a sample and not the characterization of individual aggregates. In order to reliably detect and quantify individual A-beta aggregates, ELISA-based methods also lack the necessary sensitivity.

The disease-specific protein aggregates mentioned could be a direct biomarker for the respective disease. However, diagnostic detection is a technical challenge, since these proteins are also produced in healthy organisms. Therefore, a possible detection method must be insensitive to a high excess of normally folded, monomeric protein.

In DE 10 2011 057 021 A1, a method for the characterization and quantification of pathogenic aggregates or oligomers in tissues and body fluids has been described.

However, disadvantageous for that is that biopsies are often necessary for the examination of tissues and body fluids (e.g. for blood or cerebrospinal fluid examinations), which can be painful or at least unpleasant for the patients, and that medical personnel are usually necessary for taking the samples.

Accordingly, there is a need for further methods for the selective quantification of biomarkers for neurodegenerative diseases.

In particular, there is thus a need for methods that allow disease-specific protein aggregates to be determined simply, reliably and in a manner that is as convenient as possible for patients, in particular avoiding the need for biopsies and/or the need for the consultation of medical personnel.

It was an object of the present invention to provide methods that allow disease-specific protein aggregates to be determined simply, reliably and in a manner that is as convenient as possible for patients, in particular avoiding the need for biopsies. Furthermore, it was an object of the present invention to provide a biomarker for protein aggregation diseases, in particular AD and PD, and an ultra-sensitive method for the quantification and characterization of A-beta and alpha-synuclein aggregates. By characterizing the biomarker, i.e. determining the number, amount and/or size of this substance (biomarker) in an endogenous sample that is not a fluid or tissue, an accurate diagnosis of the disease and/or information about the course of the disease and the condition of the patient should be enabled.

Still another object of the present invention was to provide a method for the selective quantification of pathogenic aggregates causing and/or characterising a protein aggregation disease, in particular A-beta and alpha-synuclein aggregates of any size and composition, A-beta and alpha-synuclein oligomers and at the same time also small, freely diffusing A-beta and alpha-synuclein oligomers.

These and further objects, which become apparent to the person skilled in the art when considering the present invention description, are solved by the subject matter illustrated in the independent claims.

The dependent claims illustrate particularly advantageous and preferred subject matter.

An essential part of the present invention lies in the diagnosis of neurodegenerative diseases based on the analysis of stool samples. Stool samples do not constitute a biopsy or a body fluid. The advantage of taking a stool sample is that it is non-invasive and therefore painless. Furthermore, stool samples can be collected without the presence of a doctor. Also, stool samples can be sent for laboratory analysis without the need for a patient to visit a doctor or laboratory in person.

Unexpectedly, it has been found in the context of the present invention that disease-specific protein aggregates can be determined simply, reliably and in the most convenient manner for patients, in particular avoiding the need for biopsies and/or the need for consultation of medical personnel by examining stool samples.

The diagnostic analysis of stool samples in neurodegenerative diseases used in the present invention was not obvious to those skilled in the art, because neurodegenerative diseases are primarily considered to be diseases of the brain and therefore only cerebrospinal fluid or blood and its components have been analysed so far.

The measurement of a biomarker based on the diagnostic analysis of stool samples has not yet been described in the literature for neurodegenerative diseases and is also not in use and thus represents a novelty.

Subject matter of the present invention therefore are in particular methods for the selective quantification of protein aggregates in stool samples as biomarkers for neurodegenerative diseases.

Subject matter of the present invention is also a method for the selective quantification and/or characterization of A-beta or alpha-synuclein aggregates in stool samples comprising the following steps:
a) providing a substrate on which capture molecules are immobilised,
b) applying the stool sample to be tested to the substrate,
c) adding probes labelled for detection, which by specific binding to A-beta or alpha-synuclein aggregates mark these, and
d) detection of the marked aggregates by means of high spatial resolution in which each event is determined in front of the respective background.

Characterization of the A-beta or alpha-synuclein aggregates or A-beta or alpha-synuclein oligomers means, in the context of the present invention, determination of the shape, size and/or composition.

In the sense of the present invention, the term A-beta or alpha-synuclein oligomers denotes both A-beta or alpha-synuclein aggregates as well as A-beta or alpha-synuclein oligomers and also small freely diffusing A-beta or alpha-synuclein oligomers. Oligomer in the sense of the invention is a polymer formed from 2, 3, 4, 5, 6, 7, 8, 9, 10, 11, 12, 13, 14, 15, 16, 17, 18, 19 or 20 monomers or multiples thereof.

In an alternative of the present invention, capture molecules are immobilized on the substrate to capture and fix the A-beta or alpha synuclein aggregates.

Preferably, anti-A-beta or alpha-synuclein antibodies are used as capture molecules.

According to the invention, a material is chosen as the substrate that has a as low as possible, non-specific binding capacity, in particular with respect to A-beta or alpha-synuclein oligomers.

In one embodiment of the present invention, a substrate made of glass is chosen.

In preferred variants of the present invention, the capture molecules are antibodies, which in variants of the present invention are immobilized not covalently, but via hydrophilic and ionic interactions between the capture molecule and the substrate surface, or covalently via poly-D-lysine, polyethylene glycol (PEG) or dextran.

In a preferred embodiment of the present invention, the glass surface is aminated for this purpose, in particular with 3-aminopropyltriethoxysilane (APTES) via the gas phase with toluene and under an argon atmosphere. Subsequently, in this embodiment, N-hydroxysuccinimide (NHS)-functionalized PEG or NHS-functionalised dextran can covalently bind to the amines of the glass surface. The immobilization of the capture molecule can be achieved in variants via the same manner by activating the carboxyl groups of PEG or dextran via EDC and NHS. The activated carboxylic acid thus covalently binds amine groups of the capture molecule to the glass surface. Remaining activated carboxylic acid groups can be inactivated via a quenching step.

In a further embodiment of the invention, the immobilization of the capture molecules can be achieved via bioaffine systems such as protein-G/A, His-tag or biotin-avidin, for example by coating the glass surface with protein-G/A, which can subsequently bind specifically, for example, to anti-mouse antibodies as a capture molecule.

The anti-A-beta or alpha-synuclein antibodies specifically bind an epitope of the A-beta or alpha-synuclein aggregates. In an alternative of the present invention, the epitope has an amino acid sequence of the amino-terminal portion of the A-beta peptide selected from the subdomains A-beta 1-11, A-beta 3-11, or pyroGluA-beta 3-11, e.g., the human N-terminal epitope having the following sequence: DAEFRHDSGYE (1-11).

For both covalent and non-covalent binding based coating, a blocking step is preferably performed after incubation with the capture molecule to minimize non-specific binding of the stool sample to still free glass surface. For this purpose, the substrate coated with capture molecules is wetted with suitable solutions known to the skilled person. In a preferred variant, a solution of bovine serum albumin (BSA) is used for this purpose.

In variants of the present invention, the stool sample to be measured and pre-treated is incubated on the substrate thus prepared.

In one variant of the present invention, a pre-treatment of the stool sample is done by one or more of the following methods:
heating (to a temperature up to the boiling point of the sample)
one or more freeze-thaw cycles,
dilution with water or buffer,
treatment with enzymes, e.g. proteases, nuclease, lipases,
centrifugation,
precipitation,
competition with probes to displace any anti-A-beta or alpha-synuclein antibodies present.
Homogenization and separation, preferably by centrifugation, of the solid from the liquid components of the stool sample with the addition of water or one or more aqueous buffer solutions.

Common buffer solutions are suitable for homogenization. Examples for these are Tris-buffered saline (TBS) or phosphate-buffered saline (PBS), which are commercially available as standard solutions. These buffer solutions may in variants be mixed with BSA.

In preferred variants of the present invention, the buffer solutions contain antimicrobially active substances in addition to the buffer substance. In particular, these are antibacterially and antifungally active substances. Examples of such substances are, inter alia, azides such as sodium azide, isothiazoline derivatives such as 2-methyl-4-isothiazoline-3-one or 5-chloro-2-methyl-4-isothiazoline-3-one, or combinations of these substances. An example of a commercially available product is ProClin™ 300 from Sigma-Aldrich (containing 2-methyl-4-isothiazolin-3-one and 5-chloro-2-methyl-4-isothiazolin-3-one).

A particularly preferred variant of the present invention is to homogenize the stool sample in a first buffer of TBS with ProClin 300 and BSA, then centrifuge and subsequently dilute the supernatant in a second buffer of TBS or PBS with BSA, detergent and sodium azide.

In a further step, A-beta or alpha-synuclein aggregates are marked by probes, which are labelled for later detection.

In one variant of the present invention, anti-A-beta or alpha-synuclein antibodies are used as probes. Capture molecules and probes may be identical.

In one embodiment of the present invention, capture molecules and probes differ. For example, different anti-A-beta or alpha-synuclein antibodies may be used as capture molecules and probes.

In another embodiment of the present invention, same capture molecules are used. In an alternative of the present invention, identical probes are used.

However, different molecules may be used as capture molecules, like e.g. different anti-A-beta or alpha-synuclein antibodies. Capture molecules can be specific amino acid sequences of the A-beta or alpha-synuclein peptide, e.g. A-beta 1-40/42, pyroGluA-beta 3-40/42 or pyroGluA-beta 11-40/42.

Likewise, different molecules can be used as probes, like e.g. different anti-A-beta or alpha-synuclein antibodies.

For subsequent quality control of the surface, e.g. uniformity of the coating with capture molecules, capture molecules labelled with fluorescent dyes can be used. For this purpose, a dye is preferably used that does not interfere with the detection. By that a subsequent checking of the structure becomes possible as well as a standardisation of the measurement results.

It is also possible to produce sample containers in stock in this way, which are thus already primed by surface coating and fixation of capture molecules and then only have to be used for the examination of a stool sample; it is therefore not absolutely necessary to produce the substrates immediately before each stool sample analysis.

In the context of the present invention, it is also possible for stool samples to be stored or frozen prior to testing. Thus, it is possible to perform analyses even on old samples.

In one embodiment of the present invention, anti-A-beta antibodies that specifically bind to the N-terminal epitope of the A-beta peptide or alpha-synuclein antibodies that bind to phosphorylated serine 129 to alpha-synuclein are used as probes.

For detection, the probes are labelled such that they emit an optically detectable signal selected from the group consisting of fluorescence, bioluminescence and chemiluminescence emissions. Similarly, the probes may be configured such that the optically detectable signal is a change in their absorption spectrum.

In an alternative embodiment, the probes are labelled with dyes. Preferably, these are fluorescent dyes.

In one embodiment of the present invention, at least 2, 3, 4, 5, 6 or more different probes are used. The probes may differ both in terms of their specific binding to the A-beta or alpha-synuclein aggregates as well as in terms of their different labelling by, for example, fluorescent dyes.

Probes that are suitable for FRET (Fluorescence Resonance Energy Transfer) as detection can also be combined with each other.

The use of several, different probes which are characterized by different fluorescent dyes increases the specificity of the correlation signal obtained during the measurement. In addition, by this also the masking out of A-beta or alpha-synuclein monomers is made possible. In particular, the detection of A-beta or alpha-synuclein monomers can be excluded if the probe and capture molecule are identical, or both detect an overlapping epitope.

In one embodiment of the present invention, probes are used which are specific for a particular A-beta or alpha-synuclein aggregate species, like e.g. A-beta (x-40), A-beta (x-42) or pyro-glutamate-A-beta (3-x), pyro-glutamate-A-beta (11-x) or alpha-synuclein phosphorylated on serine 129. X is an integer, natural number between 1 and 40 or 42, respectively, wherein the skilled person determines the length of the sequence to be used based on his knowledge of the sequence of the A-beta peptide.

In a further alternative, probes may be used that are specific for particular A-beta or alpha-synuclein aggregate forms, such as A-11 or I-11.

In another alternative, A-beat or alpha-synuclein peptides labelled with fluorescent dyes can be used as probes.

In contrast to previous prior art tests, in the context of the present invention no endogenous fluids or tissues are used. In the context of the present invention, the sample to be examined is a stool sample.

The stool samples may undergo different processing steps known to the skilled person.

For this purpose, in a particularly preferred variant, the sample is homogenized in a first buffer of TBS with ProClin 300 and BSA and then centrifuged, and the supernatant is then diluted in a second buffer of TBS or PBS with BSA, detergent and sodium azide.

A subject matter of the present invention is thus also a method for determining the composition, size and/or shape of A-beta or alpha-synuclein aggregates in stool samples. Here, the above-mentioned and described method steps are used.

The detection of the marked aggregates is performed by scanning or other types of surface imaging. Preferably, the detection is performed by confocal fluorescence microscopy, fluorescence correlation spectroscopy (FCS), especially in combination with cross correlation and single particle immunosolvent laser scanning assay and/or laser scanning microscope (LSM).

In an alternative embodiment of the present invention, the detection is performed with a confocal laser scanning microscope.

In one embodiment of the present invention, a laser focus, such as used in laser scanning microscopy or an FCS (Fluorescense Correlation Spectroscopy System), is used for this purpose, as well as the corresponding super-resolution variants such as STED or SIM. Alternatively, detection can be performed by a TIRF microscope, as well as the corresponding super-resolution variants such as STORM or dSTORM.

In the detection, a high spatial resolution is advantageous. In one embodiment of the method according to the invention, so many data points are thereby collected that the detection of an aggregate in front of a background signal caused, for example, by device-specific noise or other non-specific signals or non-specific bound probes is possible. In this way, as many values are read out (readout values) as there are spatially resolved events, such as pixels. Due to the spatial resolution, each event is determined against the respective background and thus represents an advantage over ELISA methods.

In an alternative, several different probes are used in the method according to the invention. This multiplies the information, i.e. the values read out, since for each point, for each aggregate or for each detection event, separate information is obtained depending on the respective probe providing the signal. Thus, for each event, the specificity of the signal is increased. Thus, for each detected aggregate, its composition can also be determined, i.e. the type of aggregate, such as A-beta (1-40), A-beta (1-42), pyro-glutamate-A-beta (3-40/42, 11-40/42) or mixtures thereof. In principle, the number of different probes is only limited by possible interferences of the fluorescent dyes to be used. Thus, 1, 2, 3, 4 or several different probe-dye combinations can be used.

Spatially resolved pieces of information are essential for the evaluation according to the method of the invention described above. These may be, for example, the type and/or intensity of the fluorescence. When evaluating these data for all probes used and detected, according to the invention the number of aggregates, their shape, size and/or their composition is determined. Thereby information about the size of the oligomers can be obtained directly or indirectly depending on whether the particles are smaller or larger than the spatial resolution of the imaging techniques used, in one embodiment algorithms can be used for background minimisation and/or intensity thresholds can be applied.

As fluorescent dyes those known to person skilled in the art may be used. Alternatively, GFP (Green Fluorescent Protein), conjugates and/or fusion proteins thereof, as well as quantum dots may be used.

By using internal or external standards, test results are objectively comparable with each other and thus meaningful.

In one embodiment of the present invention, at least one, preferably one, internal standard or at least one, preferably one, external standard are used, in particular for the quantification of A-beta or alpha-synuclein aggregates. In variants of the present invention, both at least one, preferably one, internal standard and at least one, preferably one, external standard are used, which can further increase the accuracy.

Following the Fluorescence Intensity Distribution Analysis (FIDA), the method according to the invention is a so-called surface-FIDA (sFIDA).

By specific selection of the capture and probe molecules it can be dictated, which size the oligomers need to have in order to contribute to detection (signal).

With the method according to the invention, the precise analysis of the small, freely diffusible A-beta or alpha-synuclein aggregates is also possible. Due to their size, which is below their resolution for light microscopes, these small A-beta or alpha-synuclein oligomers could be distinguished from background fluorescence (caused e.g. by non-specifically bound antibodies) difficultly.

In addition to the extremely high sensitivity, the method according to the invention also shows linearity with respect to the number of A-beta or alpha-synuclein aggregates over a wide range.

A further subject matter of the provisional invention is the use of the small, freely diffusible A-beta or alpha-synuclein aggregates as biomarkers for the detection and identification of protein aggregation diseases, in particular AD or PD, in stool samples. The invention also relates to a method for the identification and/or detection of protein aggregation diseases, in particular AD and PD, characterized in that a stool sample from a patient is analysed using the method of the invention described above.

In one variant of the present invention, internal or external standards are used.

Such standards for the quantification of oligomers or pathogenic aggregates characterizing a protein aggregation disease or an amyloid degeneration or protein misfolding disease are characterised in that a polymer is built up from polypeptide sequences which in relation to their sequence are identical in the corresponding subregion with the endogenous proteins or have a homology of at least 50% over the corresponding subregion with the endogenous proteins characterizing a protein aggregation disease or an amyloid degeneration or protein misfolding disease, wherein the polymers do not aggregate.

As standard in the sense of the present invention is designated a generally valid and accepted fixed reference which is used to compare and determine properties and/or quantity, in particular to determine the size and quantity of pathogenic aggregates of endogenous proteins. The standard in the sense of the present invention may be used for calibrating devices and/or measurements.

In the sense of the present invention, the term “protein aggregation disease” may also include amyloid degenerations and protein misfolding diseases. Examples of such diseases and their associated endogenous proteins include: A-beta and tau protein for AD, alpha-synuclein for PD or prion protein for prion diseases, for example, such as human Creutzfeld-Jakob disease (CJD), sheep scrapie and bovine spongiform encephalopathy (BSE).

“Homologous sequences” in the sense of the invention means that an amino acid sequence has an identity with an amino acid sequence from an endogenous pathogenic aggregate or oligomer, which causes a protein aggregation disease, of at least 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99,100%. Instead of the term “identity”, the terms “homologue” or “homology” are used synonymously in the present description. The identity between two nucleic acid sequences or polypeptide sequences is calculated by comparison using the program BESTFIT based on the algorithm of Smith, T. F. and Waterman, M. S (Adv. Appl. Math. 2: 482-489 (1981)) with the following parameters for amino acids: gap creation penalty: 8 and gap extension penalty: 2; and the following parameters for nucleic acids: gap creation penalty: 50 and gap extension penalty: 3. Preferably, the identity between two nucleic acid sequences or polypeptide sequences is defined by the identity of the nucleic acid sequence/polypeptide sequence over the respective entire sequence length, as calculated by comparison using the program GAP based on the algorithm of Needleman, S. B. and Wunsch, C. D. (J. Mol. Biol. 48: 443-453), setting the following parameters for amino acids: Gap creation penalty: 8 and Gap extension penalty: 2; and the following parameters for nucleic acids Gap creation penalty: 50 and Gap extension penalty: 3.

Two amino acid sequences are identical in the sense of the present invention if they have the same amino acid sequence.

The term “corresponding subregion” of endogenous proteins is to be understood as that peptide sequence which, according to the definitions according to the invention, has an identical peptide sequence or a peptide sequence which is homologous with the indicated percentage of a monomer from which the standards according to the invention are constructed.

It is essential for the standards according to the invention that the standards do not aggregate, preferably by using monomeric polypeptide sequences which do not aggregate since the “corresponding subregion” of endogenous proteins is not responsible for the aggregation, or which do not aggregate by blocking the groups responsible for aggregation.

Aggregates in the sense of the present invention are
particles consisting of several, preferably identical, building blocks which are not covalently bonded to one another and/or
non-covalent aggregations of several monomers.

In one embodiment of the present invention, the standards have a well-defined number of epitopes covalently linked together (directly or via amino acids, spacers and/or functional groups) for the binding of the corresponding probes.

Probes in the sense of the invention are selected from the group consisting of: Antibody, Nanobody and Affibody. As shown above, different probes can be combined within the scope of the present invention.

The number of epitopes is determined by using a polypeptide sequence that is identical in sequence to that subregion of the endogenous proteins that forms an epitope or has at least 50% homology with that subregion, and thereby has the biological activity of the epitope.

In a further embodiment of the present invention, the epitopes are epitopes of the A-beta peptide selected from the subregions A-beta 1-11, A-beta 3-11 or pyroGluA-beta 3-11, e.g., the human N-terminal epitope (having the following sequence: DAEFRHDSGYE (1-11). The epitopes for alpha-synuclein are the human full-length protein.

The standard molecule according to the invention is preferably a polymer of the polypeptide sequences defined above. Oligomer in the sense of the invention is a polymer formed from 2, 3, 4, 5, 6, 7, 8, 9, 10, 11, 12, 13, 14, 15, 16, 17, 18, 19 or 20 monomers (by monomer is meant the above polypeptide sequence), or multiples thereof, preferably 2 to 16, 4 to 16, 8 to 16, particularly preferably 8 or 16, or multiples thereof.

In an alternative of the present invention, the standards are water soluble.

In an alternative of the present invention, the standards according to the invention are composed of the same polypeptide sequences.

In an alternative of the present invention, the standards according to the invention are composed of different polypeptide sequences.

In an alternative of the present invention, such polypeptide sequences defined above are aligned in a linear conformation.

In an alternative of the present invention, such polypeptide sequences defined above are strung together to form a branched oligomer according to the invention.

In an alternative embodiment of the present invention, such polypeptide sequences defined above are strung together to form a cross-linked oligomer according to the invention.

Branched or cross-linked oligomers according to the invention can be prepared by linking individual building blocks using lysine or using click chemistry.

In an alternative embodiment, the invention relates to a standard molecule comprising or composed of copies of the amino-terminal portion of the A-beta peptide selected from the subregions A-beta 1-11, A-beta 3-11, or pyroGluA-beta 3-11, e.g., the human N-terminal epitope (having the following sequence: DAEFRHDSGYE (1-11).

The amplification of the epitopes by functional groups can be carried out before or after the synthesis of the individual building blocks. A characteristic feature of the standards according to the invention is the covalent linkage of the polypeptide sequences.

The polypeptide sequences to be used in variants according to the invention can be identical to the sequence of the A-beta full-length peptide or show a homology of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% with the sequence of the A-beta full-length peptide.

Alternatively, to construct the standard molecules according to the invention polypeptide sequences which are identical to a subregion of the A-beta full-length peptide are also used, or show a homology of 50, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% with a subregion of the A-beta full-length peptide.

Essential for the sequences used according to the invention is their property of not aggregating (or aggregating only in a controlled manner according to the conditions) and/or their activity as an epitope.

In a further embodiment of the present invention, the standards are composed as dendrimers. The dendrimers according to the invention are composed of the polypeptide sequences to be used according to the invention as described above and may contain a central scaffold molecule.

In one variant, the dendrimers according to the invention contain polypeptide sequences which have a sequence which is identical to a subregion of the A-beta peptide or shows at least 50% homology to the corresponding subregion.

According to the invention, the term at least 50% homology is also to be understood as a higher homology selected from the group consisting of 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%.

Standards, advantageously with higher solubility in the aqueous than pathogenic aggregates or oligomers from endogenous proteins, are formed in one embodiment of the invention from polypeptide sequences which are identical to or have at least 50% homology with the N-terminal region of the A-beta peptide. According to the invention, the N-terminal region of an A-beta polypeptide is to be understood as the amino acid sequence A-beta 1-8, A-beta 1-11, A-beta 1-16, A-beta 3-11 or pyroGluA-beta 3-11.

A standard molecule according to the invention may contain epitopes for at least 2, 3, 4, 5, 6, 7, 8, 9, 10 or more different probes.

Epitopes characteristic of different probes can be incorporated into the standards according to the invention by using polypeptide sequences that are identical to, or have at least 50% homology with, different regions of the A-beta peptide but have the activity of the corresponding epitope.

In one embodiment, polypeptide sequences identical to or having at least 50% homology with the N-terminal region of the A-beta polypeptide are used for this purpose, as well as polypeptide sequences identical to or having at least 50% homology with the C-terminus of the A-beta polypeptide.

In one embodiment of the present invention, the standard molecules contain so-called spacers.

A spacer is understood to be a molecule that is incorporated into the standard molecule via covalent bonds and has certain physical and/or chemical properties by which the properties of the standard molecule are modified. In one embodiment of the standards according to the invention, hydrophilic or hydrophobic, preferably hydrophilic spacers are used. Hydrophilic spacers are preferably selected from the group of molecules formed from PEG, sugar, glycerol, poly-L-lysine or beta-alanine.

In an alternative of the present invention, the standards according to the invention contain (further) functional groups.

By functional groups is meant molecules that are covalently bonded to the standard molecules. In one variant, the functional groups contain biotin groups. This enables a strong covalent bond to streptavidin. Standard molecules containing biotin groups can thus be bound to molecules containing streptavidin groups. If the standard molecules according to the invention contain biotin and/or streptavidin groups, larger standards can thus be assembled or several, possibly different standard molecules, can be bound to one scaffold.

In a further alternative of the present invention, the standard molecules contain dyes for spectrophotometric determination and/or aromatic amino acids. Aromatic amino acids are, for example, tryptophan, tyrosine, phenylalanine or histidine, or selected from this group. For example, the incorporation of tryptophan enables spectrophotometric determination of the concentration of standards in solution.

A further subject matter of the present invention are dendrimers containing polypeptides which are identical in sequence in the corresponding subregion to the endogenous proteins or have a homology of at least 50% over the corresponding subregion to the endogenous proteins characterising a protein aggregation disease, for use in the examination of stool samples.

The dendrimers may comprise any of the features of the standards described above or any combination thereof.

In an alternative embodiment of the present invention, the dendrimers are

dendrimers containing a well-defined number of epitopes for covalent binding of probes,

dendrimers containing epitopes of the A-beta peptide,

dendrimers characterized in that they have a higher solubility in the aqueous than the pathogenic aggregates of endogenous proteins which characterize a protein aggregation disease,

dendrimers containing functional groups,

dendrimers containing at least one spacer molecule and/or

dendrimers containing dyes for spectrophotometric determination and/or aromatic amino acids.

The present invention further relates to a method for preparing a standard as described above for use in stool sample analyses.

In one embodiment, the standard according to the invention is prepared by peptide synthesis or recombinant methods known to those skilled in the art.

Another subject matter of the present invention is the use of a standard described above or of a dendrimer described above for quantification in stool samples of pathogenic aggregates or oligomers of endogenous proteins characterizing a protein aggregation disease.

In one embodiment of the invention, the standard is used to quantify A-beta oligomers in stool samples.

In one embodiment of the present invention, the standards according to the invention are used for calibration in sFIDA method, ELISA (sandwich ELISA) or FACS.

In another embodiment, the present invention relates to a kit for the analysis of stool samples comprising the standards according to the invention. The compounds and/or components of the kit of the present invention may be packaged in containers optionally with/in buffers and/or solution. Alternatively, some components may be packaged in the same container. Additionally, or alternatively, one or more of the components could be absorbed to a solid support, such as a glass plate, chip or nylon membrane, or to the well of a microtiter plate. Further, the kit may include instructions for use of the kit for any of the embodiments.

In an alternative embodiment of the present invention, the standards are used to quantify pathogenic aggregates or oligomers from endogenous proteins in stool samples by:

in a first step, marking the standards or the dendrimers with probes and determining the number of probes bound to the standards or dendrimers,

in a second step, pathogenic aggregates or oligomers of endogenous proteins characterizing a protein aggregation disease are marked with probes, the number of probes binding to each pathogenic aggregate or oligomer is determined,

in a third step, the number of probes binding to each standard or dendrimer from step 1 is compared with that from step 2, and

in a fourth step, thereby determining the number and size of the oligomers in the stool sample.

In one variant of the present invention, the standards of the invention, preferably dendrimers, are used for calibration of the sFIDA method. In a first step, endogenous pathogenic aggregates from stool samples, e.g. A-beta or alpha-synuclein aggregates, are immobilized on a glass surface by a capture molecule. In the case of A-beta or alpha-synuclein aggregates, an N-terminal capture molecule can be used for this purpose. After immobilization, the aggregates are marked by two different probes. In the case of A-beta or alpha-synuclein aggregates, for example, A-beta or alpha-synuclein antibodies are used, both of which are bound via an N-terminal binding epitope. The detection probes are labelled with, preferably different, fluorescent dyes. This makes them visible in a microscope, e.g. laser scanning microscope.

According to the invention, monomer detection of endogenous polypeptides is excluded by using three different or three differently labelled probes in the test system, which bind to a similar or the same epitope. Alternatively or additionally, the detection of monomers can be excluded by not taking into account signals with a lower intensity through an intensity cut-off. Since larger aggregates have several binding sites for the two probes labelled with different dyes, monomer detection can alternatively or additionally be excluded by cross-correlation of these signals.

The standards according to the invention can be used as internal or external standards in the measurement.

Consequently, also subject matter of the present invention is a kit for the selective quantification of A-beta or alpha-synuclein aggregates in stool samples according to the method described above. Such a kit may contain one or more of the following components:
substrate made of glass, which may be coated with a hydrophilic substance, preferably a dextran layer, particularly preferably of CMD;
standard;
capture molecule;
probe;
substrate with capture molecule.

The compounds and/or components of the kit of the present invention may be packaged in containers optionally with/in buffers and/or solution. Alternatively, some components may be packaged in the same container. Additionally, or alternatively, one or more of the components could be absorbed to a solid support, such as a glass plate, chip or nylon membrane, or to the well of a microtiter plate. Further, the kit may include instructions for the use of the kit for any of the embodiments.

In another variant of the kit, the capture molecules described above are immobilized on the substrate. Additionally, the kit may contain solutions and/or buffers. To protect the dextran surface and/or the capture molecules immobilized thereon, these may be coated with a solution or buffer.

A further subject matter of the present invention is the use of the method according to the invention for diagnosis, early diagnosis and/or prognosis of AD and/or PD by means of analysis of stool samples.

A further subject matter of the present invention is the use of the method according to the invention for monitoring therapies of AD and/or PD as well as for monitoring and/or checking the efficacy of active ingredients and/or curative methods by means of analysis of stool samples. This can be used in clinical trials, studies as well as in therapy monitoring. For this purpose, samples are measured according to the method of the invention and the results are compared.

A further subject matter of the present invention is to use the method according to the invention and the biomarkers to decide whether a person is to be included in a clinical trial. For this purpose, stool samples are measured according to the method of the invention and the decision is made with respect to a threshold value.

A further subject matter of the present invention is a method for determining the efficacy of active ingredients and/or curative methods by means of the method according to the invention, in which the results of stool samples are compared with one another. The stool samples are stool from before or after, or at different times after, administration of the active ingredients or performance of the curative procedure. On the basis of the results, active ingredients and/or curative methods are selected by which a reduction of the A-beta or alpha-synuclein aggregates took place. According to the invention, the results are compared with a control which was not subjected to the active substance and/or curative method.

In the present invention thus a method is employed for the selective quantification and/or characterization of A-beta or alpha-synuclein aggregates which, after immobilization on a substrate, are specifically marked by probes, wherein the detection of the marked aggregates is carried out with a high spatial resolution. According to the invention, each event is determined in front of the respective background due to the high spatial resolution, so that only the aggregates and no non-specific signals are detected. This represents an advantage over the known ELISA methods.

In the context of the present description, Greek letters that should actually be used in designations are occasionally replaced by the full form, e.g. in the case of alpha-synuclein (SNCA), in order to increase readability and to prevent conversion errors. The meaning is not changed by this.

A particularly preferred variant of the present invention can be described by the following eight points:

Similar to a sandwich ELISA assay, yet different in very crucial respects, is the basic structure of this sFIDA assay developed according to a preferred variant of the present invention:
1. To prepare the stool sample, it is first homogenized in a buffer and then centrifuged. Subsequent enzymatic treatments of the supernatant with e.g. proteases, nucleases and lipases and possible precipitations can then be carried out if desired. Suitable dilutions resulting from this should be used for measurement in the sFIDA assay.
2. Capture molecules for protein aggregate-containing particles (e.g. anti-alpha-synuclein antibodies) are bound on a surface (preferably of glass). The capture molecules can all be identical or mixtures of different capture molecules.
3. After incubation with the capture molecule, a blocking step can take place in which the wells are treated with a blocking solution. This can optionally be followed by a further washing step.
4. The stool sample to be examined is then applied to the surface prepared in this way, preferably after previous preparation instructions. Non-specifically bound substances are removed by washing steps.
5. The protein aggregate-containing particles (e.g. alpha-synuclein aggregates) are marked with a probe useful for further detection (e.g. a fluorescent dye-bound anti-alpha-synuclein antibody that binds only a pathologically at serine 129 phosphorylated form of alpha-synuclein). Preferred fluorescent dyes could include quantum dots. Preferably, the protein aggregate-containing particles are marked with at least one further probe (e.g. a fluorescent dye-bound anti-alpha-synuclein antibody that binds only a pathologically at serine 129 phosphorylated form of alpha-synuclein). The use of several different probes coupled to different fluorescent dyes on the one hand increases the specificity of the correlation signal obtained at the end, on the other hand it also allows the masking of non-aggregated proteins (e.g. of monomeric alpha-synuclein). In particular, if one of the detection probes is identical to the capture molecule (from step 2), or both detect an overlapping epitope, the detection of non-aggregated proteins can be excluded.
6. Excess probes are removed by appropriate washing steps.
7. The detection of the marked aggregates is done by scanning (e.g. by a laser focus, such as used in laser scanning microscopy) or by other types of surface imaging (e.g. by a TIRF microscope). The higher the spatial resolution, the more data points are generated, each of which allows the detection of an aggregate in front of a background signal (e.g. by device-specific noise, non-specific signals, non-specific bound probes). In this way, not only a single readout value is generated (as would be the case with an ELISA), but as many readout values as there are spatially resolved events (for example pixels). By using several different probes (see step 3), this information is even multiplied and for each point, for each aggregate or for each detection event the information can be obtained separately as to which of the probes provides signals there. Thus, for each event, the specificity of the signal can be increased.
8. For evaluation, the spatially resolved information (e.g. fluorescence intensity) of all probes used and detected is used to determine, for example, the number of aggregates, their size and their composition. In variants of the present invention, algorithms for background minimisation may also be used and/or intensity thresholds also be applied for further evaluation.
9. Standards, both internal and external or a combination of internal and external, may be used to make the test results comparable with each other, particularly across distances, times and experimenters.

### DESCRIPTION OF THE FIGURES

FIG. 1 shows diagrammatically the results of the measurements on or examinations of stool samples from three Alzheimer patients on the one hand and five control patients on the other hand as described in Example 9a).

FIG. 2 shows diagrammatically the results of the measurements on or examinations of stool samples from four Parkinson patients on the one hand and five control patients on the other hand as described in Example 9b).

### EXAMPLES

1. Preparation of Substrate for Covalent Bonding

The amination of the glass surface was carried out with APTES and toluene. For this, 250 μl APTES were dissolved in 5 ml toluene in a crystallizing dish and placed exactly in the middle of the desiccator. A glass plate was then removed from the packaging and placed upside down in the desiccator. After drawing vacuum for approx. 10 minutes, the desiccator was flooded with argon via a balloon. After an incubation period of 1 hour, the crystallizing dish was removed and the plate was dried with the vacuum pump attached for 2 hours. The preparation after amination was not paused because the amination is not stable.

For the covalent coating with dextran, the glass surface was aminated in advance as described above. One mg/ml CMD was activated with 200 mM EDC and 50 mM NHS in 0.1 M MES pH 3.5 and added to the glass plate and incubated for 30 minutes at room temperature. Subsequent washing with water five times removed unbound dextran.

For the covalent coating with PEG, the glass surface was aminated in advance as described above. Bifunctional PEG (MW 3400 Da, NHS/COOH) was used as spacer. A 4 mM PEG solution was prepared for this purpose. First, 20 μl of PBS was added to each well and then 20 μl of the prepared PEG solution was added accordingly. After an incubation period of 1 hour at room temperature, the wells were washed five times with water. For quenching, a 10 mM ethanolamine solution was prepared and 20 μl was pipetted into each well. After a 15 minute incubation period, the wells were washed five times with water. Activation of the carboxylic acid group was performed using 20 μl of a solution consisting of 200 mM EDC and 50 mM NHS in 0.1 M MES. Following the 30 minute incubation, the wells were washed five times with water.

1a. No Pretreatment is Necessary for Non-Covalent Coating of the Glass Surface with Capture Molecule.

2. Immobilization of Antibodies as Capture Molecules on the Coated Substrate (Covalent Binding).

The solution of antibodies was added to the wells and incubated for 1 hour at room temperature. By the activation of the carboxylic acid groups of PEG and dextran as described above, the amine groups of the capture molecules were linked to PEG or dextran and thus covalently bound to the glass surface. To quench the unoccupied activated carboxylic acid groups, a 10 mM ethanolamine solution in water was used and incubated in the wells for 15 minutes.

For non-covalent binding, the capture antibody was dissolved in a suitable buffer, in this case PBS or carbonate. The concentration of 2.5 μg/ml of capture antibody used depended on the biomarker and was determined in advance by various titration experiments. Incubation was overnight at 4° C.

After the capture antibody incubation, unbound antibodies were removed by washing five times with TBS and 0.05% Tween® 20 (non-ionic surfactant; a polyethylene glycol sorbitan fatty acid ester) and washing five times with TBS.

3. Blocking

For both covalent as well as non-covalent binding, a blocking step occurred after incubation with the capture antibody. For this, BSA, casein or milk powder were dissolved in TBS at a concentration between 0.5-5.0% and incubated for 90 minutes at room temperature in the wells.

Alternatively, antibacterial and antifungal substances such as ProClin 300 or sodium azide can be added to the blocking solution. Subsequently optionally another washing step (washing five times with TBS and 0.05% Tween 20 and washing five times with TBS) can be done.

4. Immobilization of A-Beta or Alpha-Synuclein Aggregates on the Pre-Treated Substrate

The samples to be measured were incubated on the substrate for 1 hour at room temperature (20° C.) or 37° C. and then washed five times with TBS.

5. Linking the Probes with Fluorescent Dye to Label them

The detection probes, here Nab228 (anti-A-beta 1-11) or 211 (anti-alpha-synuclein 121-125) or EP1536Y (anti-phospho-alpha-synuclein (S129)) were each labelled with CF dyes, here CF633 succinimidyl ester and CF488A succinimidyl ester. The purification was carried out by size exclusion chromatography as known to the person skilled in the art.

6. Marking of the Aggregates with the Probes

The amount of antibody used depends on the desired degree of marking and a high signal-to-noise ratio, which was titrated in advance within the assay development. The probes were added and incubated for 1 hour at room temperature in the dark. Unbound probes were removed by washing five times with TBS.

7. Detection of the Aggregates and Measurement of the Samples.

The measurement was performed here with a Celldiscoverer 7 (Carl Zeiss, Jena, Germany) equipped with a confocal laser scanning microscope or a TIRF microscope (Leica, Wetzlar, Germany). The fluorescence intensity of an area of 1000×1000 pixels (TIRF) or 2752×2208 or 512×512 pixels (Celldiscoverer 7) was determined. Since different probes were used, a colocalization analysis was performed (test where measured values of both probes occurred at the same location (in this case a radius of approx. 10 nm); e.g. a red emitting fluorophore and a green emitting fluorophore can be observed). In order to obtain representative values, several areas of the glass surface (4 wells) were measured. The measurement was performed using ZEN software from Carl Zeiss or LAS AF software from Leica.

8. Preparation of Stool Samples

The stool samples can undergo different preparation steps known to the person skilled in the art.

Here, the sample was homogenized in a first buffer of Tris-buffered saline (TBS) with ProClin 300 and BSA and then centrifuged, and the supernatant was subsequently diluted in a second buffer of TBS or PBS with BSA, detergent and sodium azide.

9. Analysis of Stool Samples

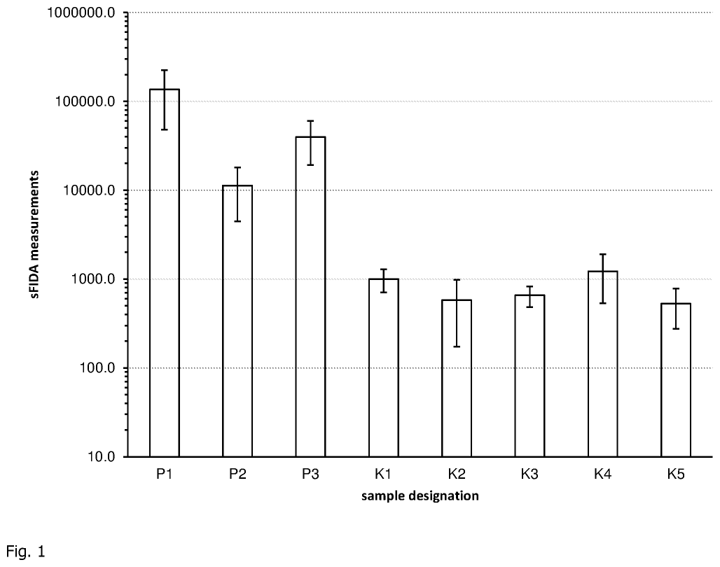

Two studies were carried out; one in relation to Alzheimer's patients and one in relation to Parkinson's patients.
a) Eight stool samples from different patients were analyzed with the method according to the invention. The stool samples were each from three Alzheimer's patients and five control patients (healthy with regard to protein aggregation diseases, different ages). The results are summarized in Table 1. Also in FIG. 1.

TABLE 1

sFIDA measurements

| sample | average of measurements | standard deviation |
|---|---|---|
| P1 | 136082.9 | 88274.6 |
| P2 | 11263.6 | 6799.7 |
| P3 | 39574.7 | 20329.0 |
| K1 | 995.1 | 289.0 |
| K2 | 578.3 | 404.5 |
| K3 | 655.1 | 171.0 |
| K4 | 1221.8 | 686.4 |
| K5 | 527.8 | 253.5 |

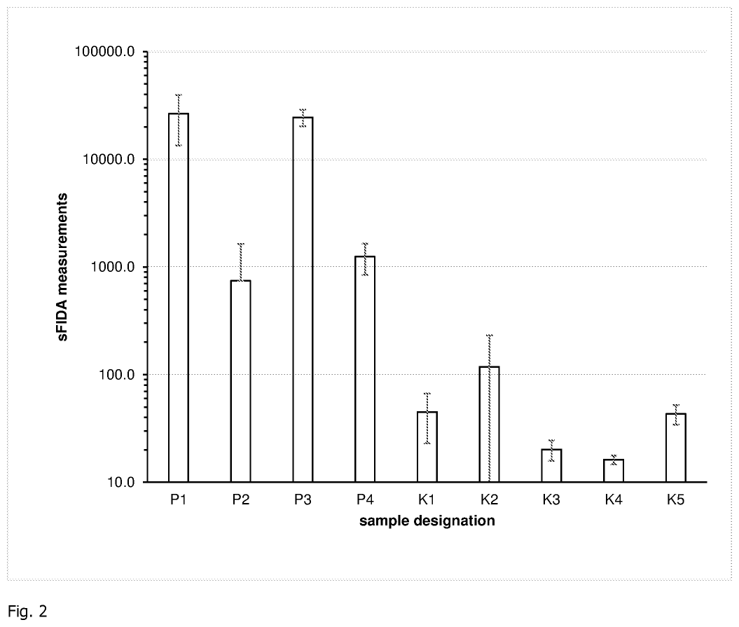

b) Nine stool samples from different patients were analyzed with the method according to the invention. The stool samples were each from four Parkinson's patients and five control patients (healthy with respect to protein aggregation diseases, different ages). The results are summarized in Table 2. Also in FIG. 2.

TABLE 2

sFIDA measurements

| sample | average of measurements | standard deviation |
|---|---|---|
| P1 | 26470.6 | 13028.8 |
| P2 | 742.6 | 891.7 |
| P3 | 24470.5 | 4188.5 |
| P4 | 1245.8 | 400.4 |
| K1 | 44.9 | 21.8 |
| K2 | 118.1 | 114.0 |
| K3 | 20.1 | 4.3 |
| K4 | 16.2 | 1.5 |
| K5 | 43.1 | 8.9 |

The results prove that a clear distinction between the groups is possible. The average of alpha-synuclein oligomers in the Alzheimer's or Parkinson's group each was significantly higher than in the respective control groups. It was therefore possible to draw conclusions about the health status of the patients from the test results of the stool samples.

1.-16. (canceled)

17. A method for the selective quantification and/or characterization of A-beta or alpha-synuclein aggregates in a stool sample, wherein the method comprises:
(a) providing a substrate on which capture molecules are immobilized,
(b) applying a stool sample to be tested to the substrate,
(c) adding probes labelled for detection, which by specific binding to A-beta or alpha-synuclein aggregates mark these, and
(d) detection of the marked aggregates by high spatial resolution in which each event is determined in front of the respective background.

18. The method of claim 17, wherein a pre-treatment of the stool sample is carried out.

19. The method of claim 17, wherein the stool sample is pre-treated by homogenization and separation of solid components from liquid components of the stool sample with addition of water or one or more aqueous buffer solutions.

20. The method of claim 17, wherein a substrate of glass is used.

21. The method of claim 20, wherein the substrate of glass has a hydrophilic coating of or based on dextran.

22. The method of claim 17, wherein the capture molecules are covalently bonded to the substrate or to the coating.

23. The method of claim 22, wherein the capture molecules are labelled with one or more fluorescent dyes.

24. The method of claim 17, wherein the capture molecules are anti-A-beta or alpha-synuclein antibodies.

25. The method of claim 24, wherein the capture molecules specifically bind an epitope of an A-beta or alpha-synuclein aggregate.

26. The method of claim 17, wherein A-beta or alpha-synuclein peptide-specific probes are used.

27. The method of claim 17, wherein the probes are fluorescent dye-labelled anti-A-beta or alpha-synuclein antibodies.

28. The method of claim 17, wherein two or more different probes are used.

29. The method of claim 17, wherein two or more probes labelled with different fluorescent dyes are used.

30. The method of claim 17, wherein at least one probe is an anti-A-beta or alpha-synuclein antibody which specifically binds to the N-terminal epitope of the A-beta peptide or to alpha-synuclein.

31. The method of claim 17, wherein the detection is carried out by spatially resolving fluorescence microscopy.

32. The method of claim 31, wherein detection is carried out by one of confocal fluorescence microscopy, fluorescence correlation spectroscopy (FCS), optionally in combination with cross correlation and single particle immunosolvent laser scanning assay, laser scanning microscopy (LSM) or TIRF microscopy, as well as a corresponding super-resolution variant STED, SIM, STORM, dSTORM.

33. The method of claim 17, wherein an internal or external standard is used for the quantification of A-beta or alpha-synuclein aggregates.

34. The method of claim 33, wherein the standard for quantification of A-beta or alpha-synuclein aggregates is a polymer composed of polypeptide sequences which with respect to their sequence are identical to that subregion of the endogenous proteins which forms an epitope or have a homology of at least 50% over the corresponding subregion with those endogenous proteins which cause a protein aggregation disease or an amyloid degeneration or protein misfolding disease, and wherein the polymers do not aggregate and the polypeptide sequence has a biological activity of the epitope.

35. A method for determining the efficacy of active ingredients and/or curative methods for the treatment of Alzheimer's disease or Parkinson's disease, wherein the method comprises carrying out the method of claim 17 and comparing the active ingredients and/or curative methods with respect to their effect on A-beta or alpha-synuclein aggregate formation, those active ingredients and/or curative methods which show lower A-beta or alpha-synuclein aggregate formation compared to a control being selected.

36. A method for making a decision regarding the inclusion of an individual in a clinical trial or test, wherein the method comprises carrying out a quantification and/or characterization of A-beta aggregates according to the method of claim 17 and comparing a measured value with a threshold value.
